(19) United States
(12) Patent Application Publication
(10) Pub. No.: US 2008/0234760 A1
(43) Pub. Date: Sep. 25, 2008

(54) Bone screw apparatus and related methods of use

(76) Inventor: Zbigniew Matulaniec, Avon, CT (US)

Correspondence Address: REINHART BOERNER VAN DEUREN S.C.;ATTN: LINDA KASULKE, DOCKET COORDINATOR, 1000 NORTH WATER STREET, SUITE 2100, MILWAUKEE, WI, 53202, US

(21) Appl. No.: 12/077,985

(22) Filed: Mar. 24, 2008

Related U.S. Application Data
(60) Provisional application No. 60/919,765, filed on Mar. 23, 2007.

(51) Int. Cl. A61B 17/04

(52) U.S. Cl. 606/309; 606/301

(57) ABSTRACT

Bone screws and related methods, as can optionally be used to affect the density of bone used therewith.

This application claims priority benefit from provisional application Ser. No. 60/919,765 filed on 23 Mar. 2007, the entirety of which is incorporated herein by reference.

### BACKGROUND OF THE INVENTION

Bone screws are used in the medical arts for treating bone fractures, for attaching corrective devices such as plates to a bone or a group of bones, for anchoring prostheses such as dentures to bone or for other tasks involving the redistribution of bone tissue. Numerous previous designs have addressed these needs but have left much room for improvement. In general, there is a need to firmly and permanently anchor the screw into bone tissue without cracking or further damaging the bone.

U.S. Pat. No. 5,964,768 (“'768 patent”), to Huebner, which is hereby incorporated by reference, includes a useful discussion of prior art bone screws and describes a bone screw having a continuously varying pitch. Pitch is the axial distance traveled by a screw with one complete turn of the screw and is defined by the axial distance between two adjacent turns of the same thread. In the screw of the '768 patent, the pitch decreases continuously from the leading end to the trailing end, and the entire body or shank of the screw is threaded. This means that as the screw is turned into the bone, succeeding threads cannot follow the precise pathway cut by earlier threads. The intent is to cause some compression of the surrounding bone tissue, thus promoting healing. Thread depth, defined as the distance between the central shank and the thread crest, varies in this screw, decreasing from the leading end to the trailing end. The crest is the point of greatest distance from the screw's longitudinal axis. Concurrently, the widths of the threads at their crests, the flat portion on these threads having greatest radial distance from the central longitudinal axis, show a decreasing trend from the leading end to the trailing end of the screw. The shank is tapered, having a smaller diameter at the leading end of the screw. This screw can leave voids in the bone near the screw surface and places the heaviest load on the weaker, more extended leading portions of the threads.

U.S. Pat. No. 5,492,442, to Lasner, describes a bone screw having a tapered shank and constant outer thread diameter. This means that the thread depth varies, decreasing from the leading end to the trailing end of the screw. This screw features a thread that thickens and curves toward the trailing end of the screw as the thread approaches the trailing end. The thread is thickened as it approaches the trailing end of the screw by increasing the angle between the distal or leading thread surface and a line normal to the screw axis. The thread apex remains sharp throughout the length of the thread and the angle formed in cross section between the two sides of the thread is kept constant along the entire length of the thread. The patentee intended to increase the pullout strength of the screw by displacing bone downward against the trailing edge of the threads with each successive turn of the screw. Turning this screw inward demands very substantial movement in the surrounding bone, and excessive disruption of the bone might not lead to optimum pullout strength for the screw.

U.S. Pat. No. 6,503,252, to Hansson, describes a bone screw with constant pitch and threads having a constant outer diameter. The thread width increases from the leading end to the trailing end of the screw, and the thread crests are flattened, displaying a region of constant radius with respect to the longitudinal axis of the screw. The screw has a tapered shank, so the thread depth decreases from the leading end to the trailing end of the screw. Leading portions of the threads have relatively large depth and carry a very substantial portion of the load.

U.S. Pat. No. 5,544,993, to Harle, which is hereby incorporated by reference, describes a bone screw having a smoothly curved surface region between adjacent turns of the thread. This departure from the sharp angles between threads and shank seen in many other designs is said to cause less stress in the surrounding bone tissue as the screw is turned.

U.S. Pat. No. 4,406,623, to Grafelmann, describes a bone screw having a tapered shank and a thread having V-shaped cutouts and an external diameter that increases from the leading end to the trailing end of the screw. The V-shaped cutouts are said to ensure that no void spaces remain in the bone after the screw is turned in. Additionally, with bone tissue filling the notched spaces in the thread path, the result is much greater resistance to rotational motion of the screw. These two factors are said to substantially improve anchoring of the screw for dental applications. The relatively large thread depths must displace substantial amounts of bone, and sharp thread edges could pose some risk of inducing cracking in the bone.

U.S. Pat. No. 4,854,311, to Steffee, which is hereby incorporated by reference, describes prior art bone screws including a bone screw having a separately threaded connecting member for attaching a corrective device or other object to the bone. The screw also features threads that lean toward the trailing end of the screw. This arrangement is said to diminish the tendency of the screw to push surrounding bone tissue radially outward as the screw is installed.

U.S. Pat. No. 4,175,555, to Herbert, describes the famous Herbert bone screw. The shaft of the screw has an unthreaded portion separating threaded portions exhibiting different pitches. The outer diameter of the threads on the trailing portion of the screw is larger than the outer diameter of the threads on the leading portion of the screw. This allows the leading threaded portion of the screw to be passed entirely through a pilot hole drilled in the first bone portion to engage the second bone portion. The larger threads of the trailing portion of the screw then engage the inner walls of the pilot hole of the first bone portion. The differing pitches of the two portions of the screw allow for the two bone fragments to be brought together as the screw is turned. The screw must be positioned properly, and there is risk that female threads on the leading portion of the screw will be stripped out if the screw is turned too far. The screw is useful for bringing portions of a fractured bone together with compressive force.

All of these screw designs represent attempts to achieve firm anchoring of the screw in bone while causing minimal further damage or cracking to bone that may already be injured or weakened with age. However, bone screws of the prior art can loosen and pull out more easily than is desirable and can cause too much stress and cracking in the surrounding bone. Threads having variable pitch can cause void spaces to be left in the bone and can cause portions of the female threads cut in the bone to strip out as the screw is turned into place. Additionally, a bone screw that is truly useful for medical purposes should appear in multiple embodiments in recognition of the variable nature of the bone material and properties and injuries to be encountered. For example, the pullout strength of a bone screw is known in the art to relate to the bone mineral density, and thread characteristics such as depth and pitch could be matched advantageously to this parameter.

### SUMMARY OF THE INVENTION

In light of the foregoing, one object of the present invention is to provide a bone screw having better gripping and biting action within bone tissue and thus better anchoring capability, thereby overcoming deficiencies and shortcomings in the art. It will be understood by those skilled in the art that one or more aspects of this invention can meet certain objectives, while one or more other aspects can meet certain other objectives. Each objective may not apply equally, in all its respects, to every aspect of this invention. As such, the following objects can be viewed in the alternative with respect to any one aspect of this invention.

Another object of the present invention is to provide a plurality of bone screws having a range of capability for compressing or dispersing bone tissue to a desired extent.

Another object of the present invention is to provide a bone screw with a reduced tendency to strip out female threads as the screw is installed, as can be accomplished through more uniform load distribution along the entire lengths of the threads.

Another object of the present invention is to provide a bone screw having a decreased tendency to crack or further injure surrounding bone tissue as it is installed.

Another object of the present invention is to provide to the medical professional a range of bone screw choices that allow the particular characteristics of the bone injury or task at hand to be taken into account to achieve optimal bone screw performance.

Other objects, features, benefits and advantages of the present invention will be apparent from this summary and descriptions of certain embodiments and will be readily apparent to those skilled in the art having knowledge of various medical conditions involving injury to or atrophy of bone tissue, methods of installing bone plates and other healing aids, methods of treating bone fractures, materials and methods useful in the design and manufacture of screws and other fasteners and engineering or mathematical methods useful in understanding the stresses and motion induced when an intruding object disrupts bone tissue. Such objects, features, benefits and advantages will be apparent from the above, as taken into conjunction with the accompanying examples, data, figures and all reasonable inferences to be drawn therefrom, alone or with consideration of the references incorporated herein.

In part, the present invention can comprise, without limitation, a bone screw comprising a shank comprising a proximal end portion and a distal end portion, and one or a plurality of threads comprising a helical configuration thereabout. Each such thread can comprise a proximal side directed toward the proximal end portion and a distal side directed toward the distal end portion. Each such thread can be imagined or considered to envelop therein a reference helical configuration of substantially constant pitch. At least one such thread can vary in a width dimension. As thread width increases, at least one of the proximal side and the distal side of each such thread can be positioned a distance from the helical reference. From another perspective, thread crest width can incrementally increase proximally and/or distally with respect to such a reference helix as a proximal and/or distal end of the thread is approached. Those skilled in the art will recognize that as one end of each such thread is approached and either the proximal and/or distal side of each such thread is moved away from a reference helix, an infinite variety of embodiments is available. For instance, in certain non-limiting embodiments, the thread crest width can be increased along the thread length by positioning both the proximal and distal sides of each such thread farther away from a reference helix, and the rate of dimensional change from the reference helix in each such direction can be substantially the same or can vary along the thread.

Regardless, the shank of any such bone screw embodiment can be substantially cylindrical, with a radius substantially constant about a linear longitudinal axis. In certain other embodiments, the shank can vary in radial dimension along such an axis. In certain such embodiments, the shank can be tapered. Without limitation, the taper of such a shank can provide for a radial dimension or diameter smaller at or about the distal end portion, as compared to the radial/diameter dimension at or about the proximal end portion. Regardless, the rate of change of such radial/diameter dimension can be constant or can vary along the length of the shank.

As discussed above, each such thread comprises a crest at a distance from the shank of the bone screw, as can be considered in terms of thread depth. In certain non-limiting embodiments, thread depth can be substantially constant along thread length. In certain other embodiments, thread depth can be varied therealong.

In part, the present invention can also comprise a method of using thread crest width to affect bone density. Such a method can comprise providing a bone screw of the sort discussed above, comprising at least one thread of increasing crest width; and inserting the screw into a bone substrate. In certain non-limiting embodiments, as illustrated herein, such a thread can comprise increased crest width and/or increased distance of the distal side of such a thread, as can be imagined or considered with respect to a reference helical configuration of substantially constant pitch toward the proximal end portion, and inserting the screw into a bone substrate can compress or compact bone substrate material. In certain other non-limiting embodiments, such a thread crest width can increase with increased distance of the proximal side of such a thread, as can be imagined or considered with respect to a reference helical configuration of substantially constant pitch toward the proximal end portion, and inserting the screw into a bone substrate can disperse bone material. In other non-limiting embodiments, such a thread crest width can increase with increased distances of both proximal and distal sides of the thread with respect to a reference helical configuration of substantially constant pitch toward the proximal end portion, wherein the distances between the two respective sides of the thread and the reference helix can be the same or different at any given point along the length of the thread and can increase at the same or different magnitudes of increment per unit of distance along the length of the thread as the proximal end portion is approached.

In part, the present invention can comprise a method for healing bone fractures. Such a method can comprise, without limitation, providing a bone screw that can have one of the configurations described above, placing the distal end of the screw into contact with one fragment of a fractured bone substrate, turning the screw through this first fragment and into at least one other fragment, and drawing the fragments of the bone one to another and/or together by causing the screw to rotate, enter the bone fragments, and simultaneously compress bone tissue.

In part, the present invention can comprise a mechanical system comprising a prosthesis or healing aid to bone. Without limitation, prostheses can include dentures, components thereof, artificial limbs and digits (i.e., appendages). Healing aids can include, without limitation, orthopdic plates, rods or other structural aids. The system can comprise, without limitation, a bone screw comprising a configuration of the sort described above, and a component thereon for removably snapping, threadedly engaging or otherwise coupling the proximal end of a screw with such a prosthesis or healing aid. Using a turning means, such as a screwdriver, Allen wrench, lug wrench or open end wrench, a screw can be inserted into a bone structure or substrate, and a prosthesis or healing aid can be coupled to the proximal end thereof.

Certain embodiments of the invention along with additional features and advantages thereof will be better understood by reference to the following detailed description with further reference to the accompanying drawings.

### BRIEF DESCRIPTION OF THE DRAWINGS

Further features of the present invention will become apparent to those skilled in the art through examination of the accompanying drawings, which are briefly described as follows:

FIG. 1a is a perspective view of a bone screw according to one embodiment of the invention, having a central shank of constant diameter.

FIG. 1b is a perspective view of a bone screw according to one embodiment of the invention, having a tapered central shank having a diameter that is wider at the trailing end than at the leading end of the screw and varies in a linear fashion with position along the longitudinal axis of the screw.

FIG. 1c is an axial view of the screw shown in FIG. 1a, showing two threads leading away from the leading end of the screw.

FIG. 2a is a side view of the screw shown in FIG. 1a.

FIG. 2b is a side view of the screw shown in FIG. 1b.

FIG. 3a is a cross-sectional view of the screw shown in FIG. 1a, showing thread pitch and depth.

FIG. 3b is a cross-sectional view of the screw shown in FIG. 1b, showing thread pitch and depth.

FIG. 4a is a partial closeup side view of a portion of a screw of the type shown in FIG. 1a, showing a thread configuration that would induce compression of the surrounding bone.

FIG. 4b is a partial closeup side view of a portion of a screw of the type shown in FIG. 1a, showing a thread configuration that would induce dispersion of the surrounding bone.

FIG. 4c is a partial closeup side view of a portion of a screw of the type shown in FIG. 1a, showing a thread configuration that would induce a mixed effect of compression and dispersion in the surrounding bone.

FIG. 5a is a cross-sectional side view and depicts the turning of the screw of FIG. 4a into bone tissue, indicating movement induced in the surrounding bone.

FIG. 5b is a cross-sectional side view and depicts the turning of the screw of FIG. 4b into bone tissue, indicating movement induced in the surrounding bone.

FIG. 6 is a partial cross-sectional side view of a bone substrate and a prosthesis, and depicts the use of a bone screw according to one embodiment of the present invention to secure a denture component to a jawbone.

### DETAILED DESCRIPTION OF CERTAIN EMBODIMENTS OF THE INVENTION

This invention comprises bone screws for use by medical professionals. The subject bone screws include but are not limited in their design to those described here. Medical professionals may choose from the designs described herein; their needs may vary depending upon the kind of repair to be made and the type of bone involved.

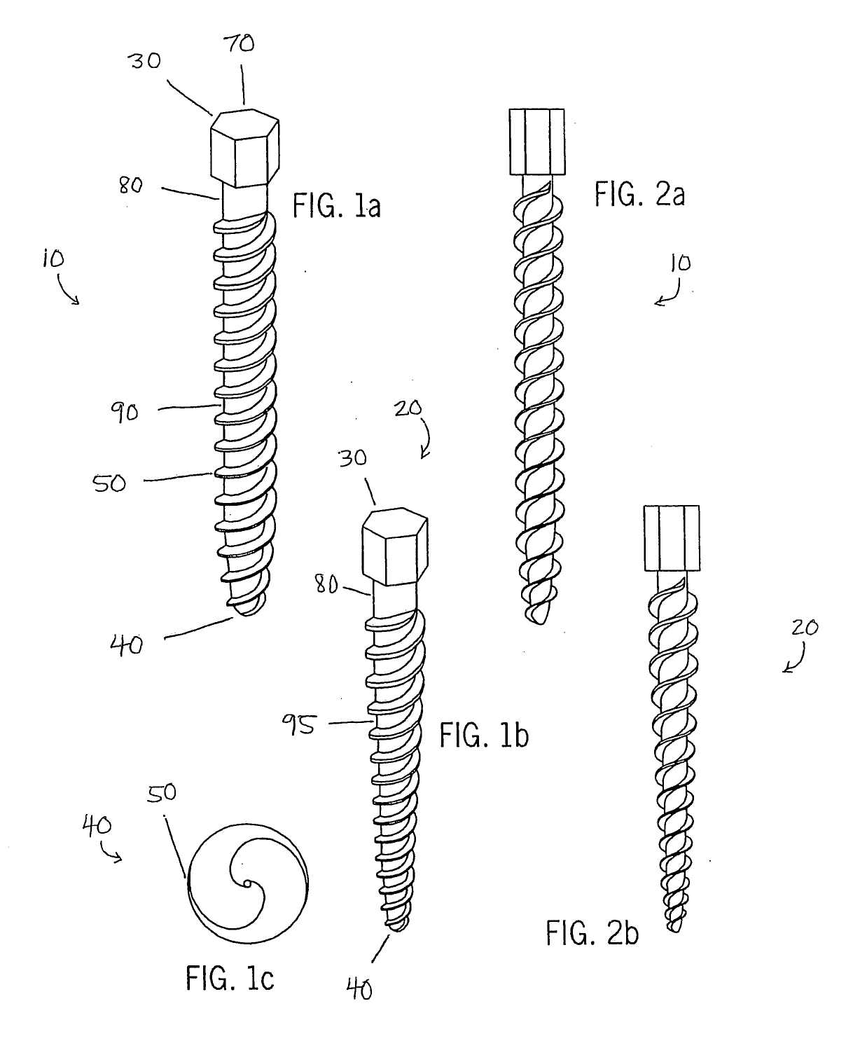

FIG. 1a depicts a bone screw comprising the distinctive features of the present invention, though the present invention is in no way limited to the particular embodiment shown. The screw 10 features a central shank 90 having a cylindrical shape with a substantially constant diameter along its length. The screw has a proximal or trailing end 30 and a distal or leading end 40. The threads 50 can protrude from and wrap around the surface of the shank 90, forming a generally helical shape. The shank 90 can exhibit near its proximal end 30 a smooth region 80 bearing no threads. The proximal end 30 can feature a recessed hexhead (not shown) for receiving an Allen wrench, a slot (not shown) for receiving the blade of a screwdriver, or a male hexhead 10 (shown) for receiving an appropriately designed lug wrench or open end wrench. The distal end 40 can taper to a point (shown) to enable a self-drilling screw to burrow through bone, thrusting bone tissue aside. Alternatively, the distal end 40 can feature a flat region enabling a self-tapping screw to achieve comparable purchase with somewhat less length in comparison with the self-drilling screw. A screw of either distal end design can be used within the present invention with or without a predrilled pilot hole.

FIG. 1b depicts, without limitation, another embodiment of the present invention. This embodiment can feature a shank 95 that can be tapered such that its radius about the longitudinal axis of the screw can vary in a substantially linear fashion with position along the longitudinal axis. The shank can have a larger radius near the region ending adjacent to the proximal end 30 and a smaller radius in the region of the shank 95 that approaches the distal end 40. This tapered screw 20 can display other features comparable to those described for the screw 10.

FIG. 1c is an axial view of the screw 10, showing an exemplary double-threaded boring tip, though bone screws of the invention can comprise, without limitation, one, two, three or more threads 50 on each screw. On screws having a plurality of threads, such threads can parallel each other. In the embodiment shown, two threads 50 originate at the distal end 40 and wrap in parallel in a helical fashion about the shank 90 and along its length, ending when they reach the smooth region 80. As is known in the art, multiple threads can help to balance the screw as it is turned into the bone, helping to prevent its destabilization through lateral movement and helping to prevent the skewing of its direction of progress.

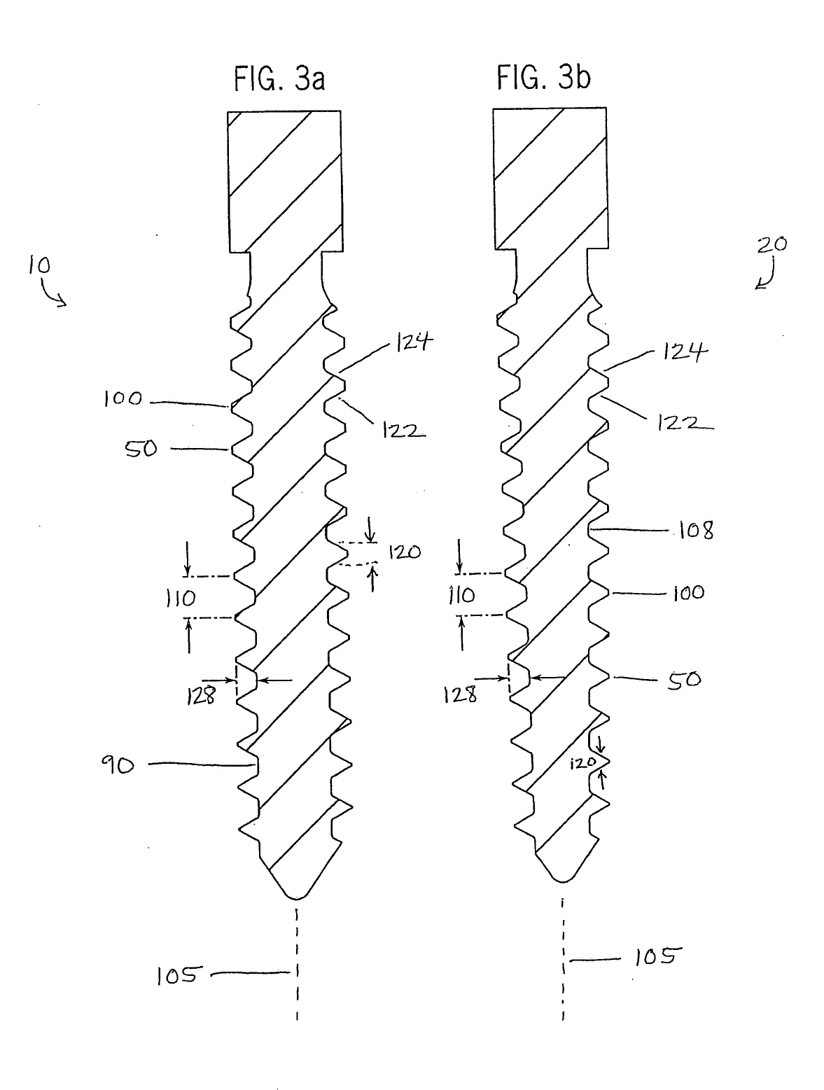

FIGS. 2a and 2b show side views of the screws 10 and 20, and FIG. 3a depicts a cross-sectional view of the screw 10. Threads 50 wind about the shank 90 in a helical fashion. Preferably, threads of the bone screws of the invention can be milled in such a way that the angles formed by the sides of the threads with the longitudinal axis of the screw are constant along the entire length of the threads. Each thread 50 exhibits a thread peak or crest 100, defined as the point at which the material of the thread reaches its greatest distance from the screw's longitudinal axis 105. In certain embodiments of the invention, the thread peaks/crests 100 remain at the same distance from the shank 90 along the entire lengths of the threads 50. Certain embodiments of the present invention comprise threads 50 that fully envelope an imaginary reference helix of constant pitch along their entire lengths. A helix of constant pitch is one exhibiting a constant axial distance between adjacent turns of the helix. Such threads can be said to exhibit constant pitch, though thread widths 120 can vary and thread sides 122 and 124 need not separately in isolation display constant pitch. Thread width 120 varies and can be determined anywhere thereon, at a point removed from shank 90 and, in particular, can be referenced with respect to thread crest 100. Without limitation, the screws of the present invention can exhibit both constant pitch and constant thread depth, where thread depth 128 refers to the distance along a shank radius between thread peaks/crests 100 and the shape defined by the shank 90.

FIG. 3b shows a cross-sectional view of the screw 20 of FIG. 1b. The threads 50 of the screw 20 can exhibit constant depth as shown by a constant radial distance from thread peaks/crests 100 to thread troughs 108. In certain embodiments of the screw 20, thread troughs 108 can be configured such that every plane intersecting the longitudinal axis 105 also intersects portions of the bottoms of the thread troughs in a line segment that is straight and parallel to the longitudinal axis 105.

In certain embodiments of the invention, the threads 50 of the bone screw encompass an imaginary helix of constant pitch 110. In these embodiments, grooves cut by leading portions of the threads are not vacated as the screw is turned farther into the bone. Configuring threads in such a manner that the angles formed in any thread cross section by thread sides 122 and 124, respectively, with a coplanar line normal to the longitudinal axis 105 remain substantially constant over the length of the threads helps to ensure that voids will not be left in the bone by the advancing screw.

Also, the threads of the screws of certain embodiments of the invention widen as the proximal end is approached, and varying the thread width in this manner makes the screw robust. These widening portions of the threads do work upon the bone and experience resistance from the bone. Thus, the load is distributed more uniformly along the length of the thread than it would be if the thread had a constant width. Many prior art screws having threads of constant width suffer from the disadvantage that the leading portions of the threads are doing most of the work and bearing most of the stress that is generated as the screw is rotated into place.

Other features of the threads of the invention can also be useful in optimizing the function of the screws. The bone screws of the invention can feature threads of constant depth 128. This means that, in certain embodiments, the distance along a radial line normal to the screw's central longitudinal axis 105 from the surface of the shape embodied by the shank 90 to the thread peak 100 is constant over the lengths of the threads 50. The advantage offered by this arrangement is that once the bone tissue volume to be occupied by the shank of the screw is displaced, additional radial forces upon the bone that might cause cracking or other undesired disruption are minimized as the screw is installed. When the trailing portion of a thread follows the groove cut by the leading portion of that thread, there is much less risk of stripping out female threads that were cut in the bone by the advancing screw. Additionally, threads of the invention can be flattened at their peaks 100, thereby avoiding the cleaving action that may be induced by the turning of a sharper peak through the surrounding bone.

In certain embodiments of the invention, a constant shank radius can provide for gentle yet effective contact with the surrounding bone. With thread widths widening as the proximal end is approached, bone tissue residing between adjacent thread turns can be pushed primarily in an axial direction, either inward or outward with respect to the surface of the bone. Because in these certain embodiments the regions between threads present no sharp corners and because compression of bone tissue in an axial direction will necessarily induce some force in radial directions, very significant grip and bite can be maintained along the entire length of the screw.

In an alternative embodiment of the invention, a slight taper can be added to the shank of the screw such that the shank becomes slightly wider near the proximal end than it appears at the distal end of the screw. This will, of course, increase forces exerted in radial directions upon the bone tissue as the screw is installed, increasing the grip of the screw in bone capable of receiving this stress without cracking. Improvement can be very significant over screws of the prior art that show radical changes in shank diameter over the length of the screw. Such radically changing shank diameters can induce undesired cracking and damage to the bone.

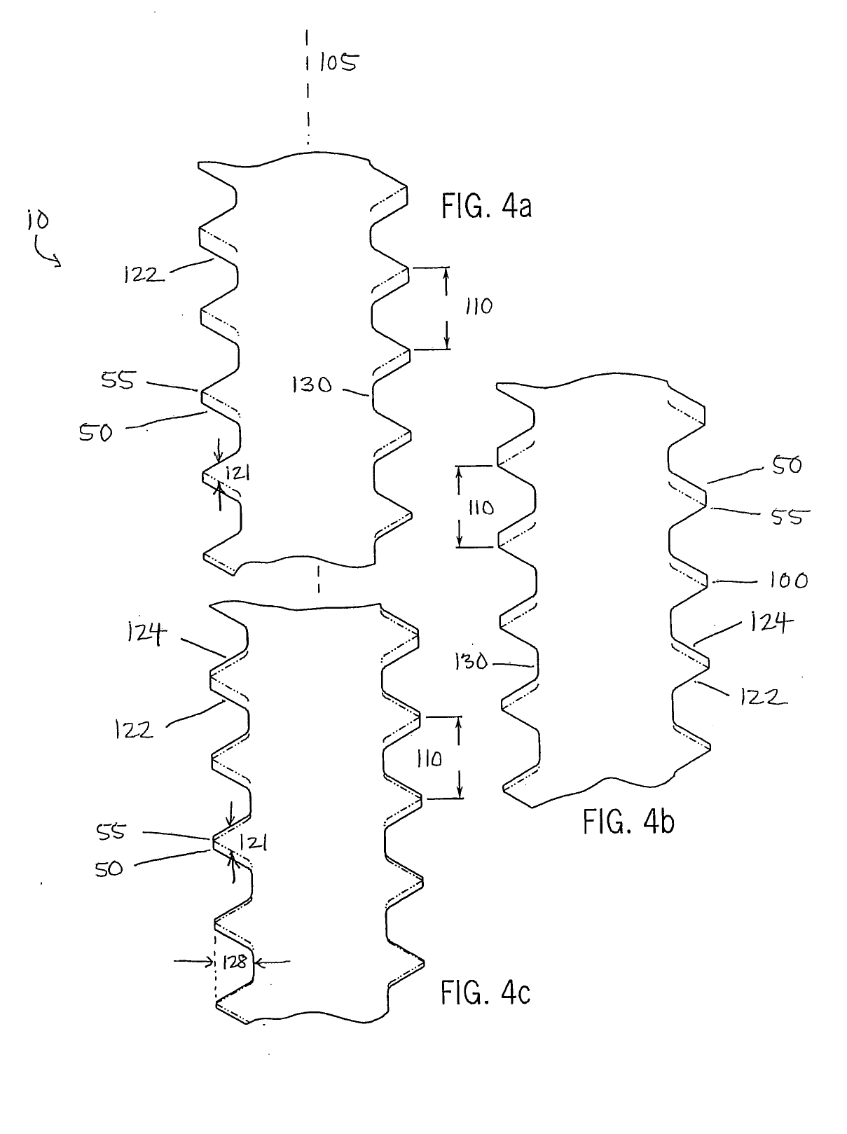

FIG. 4a depicts, without limitation, a cross section of a portion of the shank 90 of certain possible embodiments of the screw 10 of FIG. 1a. Dotted lines depict hypothetical threads 55 that symmetrically encompass an imaginary reference helix of constant pitch 110. The hypothetical threads exhibit a constant thread width 121 as measured, for example, at half height. Solid lines show the shape of threads 50 milled according to these certain embodiments of the invention; trends in thread shape may be exaggerated in FIG. 4 relative to certain embodiments for illustration purposes. As the proximal end 30 is approached while moving along the lengths of threads 50, thread width 120 can increase and the distal sides 122 of threads 50 can move progressively farther away from the imaginary helix of constant pitch 110. As threads approach the proximal end 30, thread crests 120 can become wider and more flattened. In the embodiments depicted here, the proximal sides of threads 50 can remain at a constant distance from the imaginary reference helix of constant pitch 110. The screw surface region 130 that lies between adjacent turns of the same or different threads 50 can be milled to any desired shape that is concave when viewed from the exterior of the screw. The region 130 can comprise a portion having constant radial distance from the longitudinal axis 105 along a shank surface segment parallel to said axis.

FIG. 4b depicts, without limitation, certain alternative embodiments of the invention. Again, dotted lines depict a hypothetical thread 55 having a constant width 121 and a constant pitch 110. In these embodiments, as the proximal end 30 is approached along threads 50, the proximal side 124 of the threads 50 can move progressively farther from the imaginary helix of constant pitch, while the distal sides 122 of the threads 50 can remain at a constant distance from said helix. Again, the thread peaks 100 can flatten as the threads 50 approach the proximal end 30.

FIG. 4c depicts, without limitation, certain other embodiments of the bone screw of the present invention. Again, dotted lines depict a hypothetical thread 55 of constant thread width 121, constant pitch 110 and constant depth 128. In these embodiments, both the proximal side 124 and the distal side 122 of the thread 50 can move away from the imaginary helix of constant pitch 110 as the proximal end 30 is approached. As the proximal end 30 is approached along threads 50, the proximal sides 124 and the distal sides 122 of the threads 50 can move away from said helix at the same or different rates relative to position along the threads. If the latter rates are different and nonzero as depicted in the embodiment of FIG. 4c, the geometric centers of threads 50 within successive thread cross sectional elements move farther from the imaginary reference helix of constant pitch 110 as said elements approach the proximal end 30. Depending upon the relative magnitudes of the latter rates, said geometric centers could move in either a proximal or a distal direction within the planes of said thread cross sectional elements as the proximal end 30 is approached. In these embodiments, the imaginary reference helix of constant pitch 110 is again fully enveloped by the threads 50.

Additional analogous embodiments can be constructed using the screws 20 having tapered shanks 95. These embodiments preferably comprise concave surfaces 130 which appear between threads, each comprising a “flat” region, wherein the surface of any portion of the flat region contains an infinite number of line segments that lie between adjacent thread crests and substantially parallel to the longitudinal axis 105.

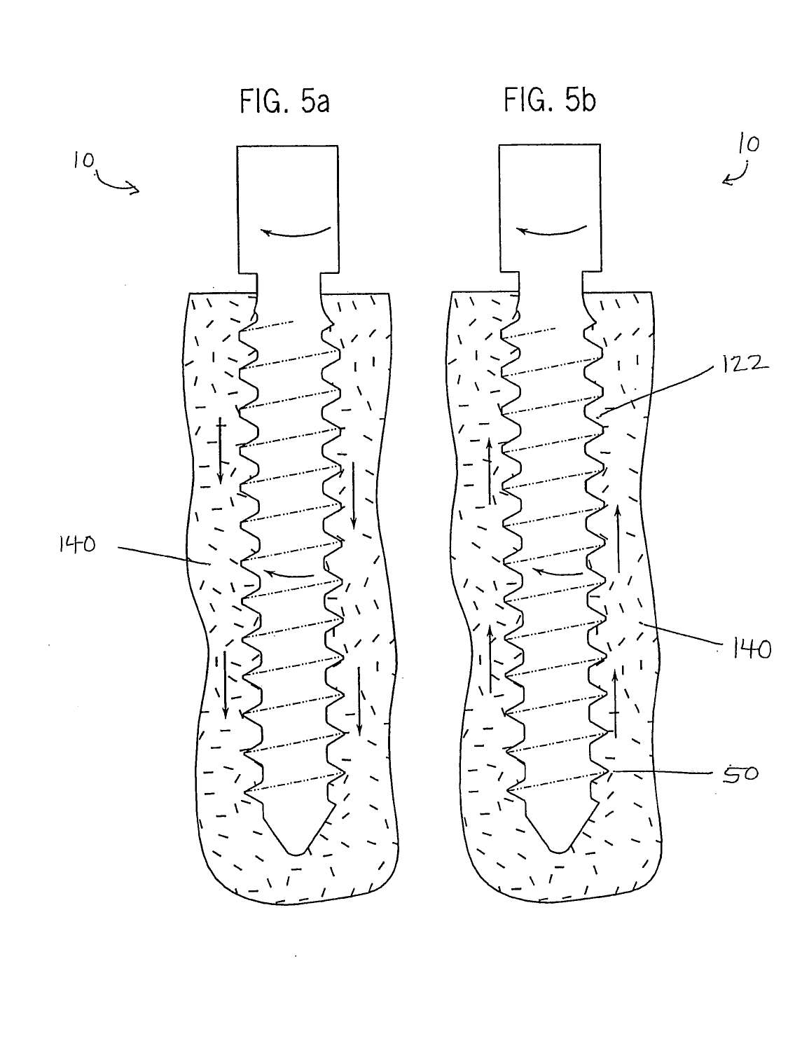

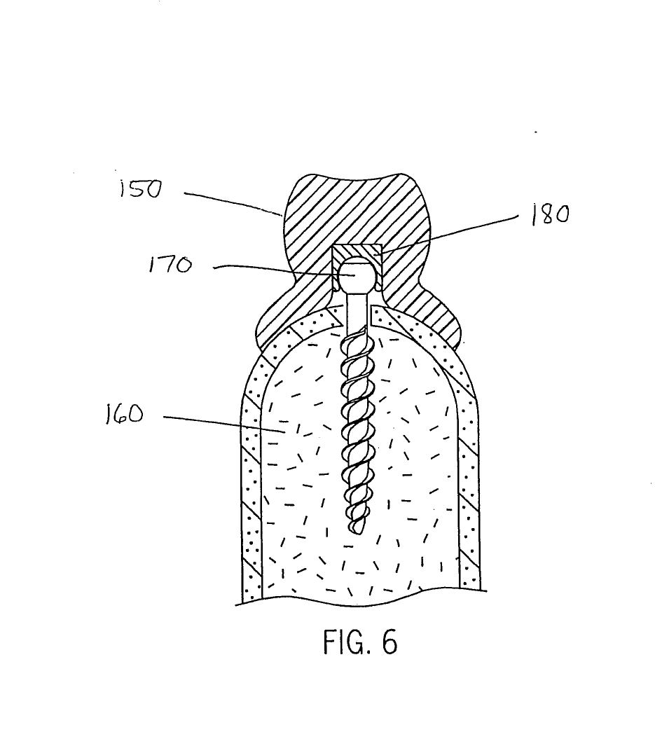

Without limitation, FIG. 5a depicts changes that can occur as the screw 10 of FIG. 4a is driven by rotation into a portion of bone 140. As illustrated, this embodiment compresses bone by pushing the bone tissue that surrounds the screw in an inward direction relative to the bone surface as the screw is driven into the bone by rotation. As the screw is thus driven inward, curves can be cut into the bone by the initial portions of the threads. As the screw is driven deeper, succeeding turns of the threads can present increasing pressure to the bottom sides of said curves as the succeeding turns follow the previously cut curves. Bone tissue can thus be pushed away from the bone surface, and the overall effect can be to compress the bone tissue.

FIG. 5b depicts, without limitation, the screw 10 of FIG. 4b in cross section as it is driven into a portion of bone 140. Again, curves are cut in the bone tissue by the initial portions of the threads 50. However, in this embodiment of the screw 10, the proximal sides 122 of the threads 50, by their movement away from the imaginary helix of constant pitch as the proximal ends 30 are approached along threads 50, can exert, relative to the bone surface, outward pressure upon surrounding bone tissue as the screw is driven deeper by its rotation. This outward pressure upon the surrounding bone tissue can tend to disperse the bone tissue.

In various other embodiments of the screw 10, both the proximal sides 124 and the distal sides 122 of the threads 50 move away from the imaginary helix of constant pitch, though they can do so at the same or different rates with respect to position along the lengths of the threads. Accordingly, relative to the bone surface, both inward and outward pressures can be applied to the surrounding bone tissue as the screws of these embodiments are driven inward by their rotation. This can achieve a mixed function of compression and dispersion of bone that can be of value in some clinical situations.

Another advantage of screws according to certain embodiments of the present invention becomes apparent from an examination of FIGS. 4 and 5. The effect of threads that widen as the proximal end of the screw is approached is to distribute the load associated with rearrangement of the surrounding bone tissue more evenly along the length of the threads. This also means that the gripping and biting action exerted by the threads upon the surrounding bone tissue will be distributed along the entire length of the screw, thus providing greater purchase and pullout strength than would otherwise be possible. Adding a taper to the shank of the screw can allow for the application of additional pressure in a radial direction with each successive turn of the screw and can be more suitable for softer bone.

By choosing from among embodiments of this invention, a medical professional can customize the directions and magnitudes of forces exerted upon bone tissue to suit the particular injury or task involved. Objectives can be to bring bone tissue together for healing, to obtain maximum pullout strength for purposes of anchoring a prosthesis, or to achieve a desired attachment task without causing breakage of brittle bones. Variables including the extent of taper of the shank, thread pitch, thread width, thread depth, the shape of the leading end of the screw, and the shapes of the valleys between adjacent thread turns can separately or in combination be varied in conjunction with thread shapes of the invention to produce screws customized for very specific purposes. Screws manufactured according to the invention can provide an improved and customizable way of fastening bone tissue to itself or a heeling or structural aid while achieving a desired extent of dispersion or compression of the bone tissue. Matching the screw to the brittleness of the bone tissue involved can help to achieve maximum anchoring effectiveness.

Considerable flexibility remains for engineers who design screws to be made according to the invention. Such screws can be made using materials, procedures and manufacturing techniques known to those skilled in the art made aware of this invention. Screws according to the invention can be machined or made from any biocompatible material, including but not limited to titanium, cobalt, medical grade stainless steel, chromium alloys, carbon fiber materials, resins, plastics, ceramics and various polymers. Suitable polymers can include but are not limited to polyether ether ketone (PEK), polyetherketoneketone (PEKK) and polyetherketoneetherketoneketone (PEKEKK). Polymers can be used with or without reinforcement by fibers composed of carbon or glass.

Applications for bone screws of the invention are many. For example, bone screws can be used to secure dentures to a jaw bone, as shown in U.S. Pat. No. 6,692,254, to Kligerman et al., which is hereby incorporated by reference. Dental implants place very demanding loads upon the screws that anchor them. Certain embodiments of the bone screws of the invention can take advantage of the relatively high strength and relatively high density of the jaw bone, making the use of higher insertion torques and the corresponding achievement of higher pullout strength possible, thereby overcoming deficiencies and shortcomings in the art. Fractured bones including tibias, femurs, ankles, wrists, hips, shoulders, and others can be treated by insertion of intramedullary nails of various lengths, such nails being anchored in place using cross-locking screws that insert through holes drilled in the nails in directions perpendicular to the long axes of the nails. Certain embodiments of the bone screws of the present invention can be expected to improve the stabilization of bone by means of intramedullary nails by achieving better immobility and pullout strength. The use of bone screws to secure intramedullary nails in the repair of bone fractures is described in U.S. Pat. No. 6,508,820, to Bales, which is hereby incorporated by reference. Bone screws can be used to secure plates to bone, as with the spinal plate of U.S. Pat. No. 7,166,111, to Kolb and Fanger, which is hereby incorporated by reference. Screws threaded according to the invention can be used for a myriad of other anchoring tasks, such as with the pedicle screw assembly of U.S. Pat. No. 7,163,539, to Abdelgany and Markworth, which is hereby incorporated by reference.

FIG. 6 shows, without limitation, one potential application of screws of the present invention. Said screws can be used to mount dentures 150 to the jaw bone 160 of a dental patient. Screws having a ball-shaped or other suitable fitting 170 at the proximal end can be turned into the bone of the jaw. Subsequently, a dental prosthesis featuring fitting receptacle members 180 which are designed to receive the fittings 170 can be snapped into place. Such a design can offer the advantage that the prosthesis can be removed and snapped back into place as necessary. Various other prostheses, healing aids and receptacle members thereof together with bone screw fittings useful in conjunction therewith would be known to those skilled in the art made aware of this invention.

Those skilled in the art will appreciate that the scope of the invention is not limited to the embodiments specifically described herein. Various modifications in thread shapes and in other dimensions of the subject screws may be possible without departing from the spirit and scope of the invention as depicted herein.

What is claimed is:

1. A bone screw comprising a shank comprising a proximal end portion and a distal end portion; and at least one thread about said shank, said thread comprising a helical configuration, each said thread comprising a proximal side, a distal side and a crest therebetween, said thread sides enveloping a reference helix of constant pitch, at least one said thread comprising a width dimension varied along the length dimension of said thread.

2. The bone screw of claim 1 wherein one said thread width dimension increases at least one of proximally and distally, said increase indicated by said reference helix, at least one of said proximal and distal thread sides increasing in distance from said reference helix.

3. The bone screw of claim 2 wherein said rate of width increase is substantially constant.

4. The bone screw of claim 2 wherein said thread crest dimension increases distally along said thread.

5. The bone screw of claim 2 wherein said thread crest width increases proximally along said thread.

6. The bone screw of claim 1 wherein said shank has a configuration selected from substantially cylindrical and tapered; and one said thread width dimension increases at least one of proximally and distally, said increase indicated by said reference helix, at least one of said proximal and distal thread sides increasing in distance from said reference helix.

7. The bone screw of claim 6 wherein one said thread has a depth dimension substantially constant along said length.

8. The bone screw of claim 6 wherein said thread crest dimension increases distally along said thread.

9. The bone screw of claim 6 wherein said thread crest width increases proximally along said thread.

10. The bone screw of claim 1 coupled to one of a prosthesis and a healing aid.

11. The bone screw of claim 10 inserted into a bone substrate.

12. The bone screw of claim 1 comprising a plurality of said threads.

13. A mechanical system comprising a bone screw of claim 1 coupled to one of a prosthesis and healing aid.

14. The system of claim 13 wherein said thread width dimension increases substantially constantly along said thread, said thread width increasing at least one of proximally and distally, said increase indicated by said reference helix, at least one of said proximal and distal thread sides increasing in distance from said reference helix.

15. The system of claim 14 wherein said shank has configuration selected from substantially cylindrical and tapered.

16. The system of claim 15 wherein said thread crest dimension increases distally along said thread.

17. The system of claim 15 wherein said thread crest dimension increases proximally along said thread.

18. The system of claim 10 wherein said prosthesis is selected from a denture component and an artificial appendage component; and said healing aid is selected from orthopedic plates and orthopedic rods.

19. A method of using a bone screw thread crest width to affect bone density, said method comprising;
providing a bone screw of claim 1, said bone screw comprising at least one thread of increasing crest width; and
inserting said bone screw into a bone substrate.

20. The method of claim 19 wherein said shank has a configuration selected from substantially cylindrical and tapered; and one said thread width dimension increases at least one of proximally and distally, said increase indicated by said reference helix, at least one of said proximal and distal thread sides increasing in distance from said reference helix.

21. The method of claim 20 wherein said crest width increases distally, and insertion of said bone screw compresses said bone substrate.

22. The method of claim 21 wherein said substrate comprises fractured fragments, and said bone screw is inserted into first and second fragments of said fractured bone substrate, said insertion drawing said bone fragments one to another.

23. The method of claim 20 wherein said crest width increases proximally along said thread, and insertion of said bone screw disperses said bone substrate.

24. The method of claim 20 wherein said shank is tapered.

25. The method of claim 19 wherein said bone substrate is selected from jaw, arm, leg, ankle, wrist, hip and shoulder bones.
